(19) United States
(12) Patent Application Publication
(10) Pub. No.: US 2011/0111115 A1
(43) Pub. Date: May 12, 2011

(54) REBAUDIOSIDE A POLYMORPHS AND METHODS TO PREPARE THEM

(76) Inventors: Jingang Shi, Beijing (CN); Yunlong Feng, Tianjin (CN); Chenghai Zhao, Beijing (CN); Hansheng Wang, Shanxi (CN)

(21) Appl. No.: 12/939,370

(22) Filed: Nov. 4, 2010

Related U.S. Application Data
(60) Provisional application No. 61/258,799, filed on Nov. 6, 2009.

(51) Int. Cl. A23L 1/236; C07H 15/24

(52) U.S. Cl. 426/658; 536/18.1

(57) ABSTRACT

The invention describes substantially pure rebaudioside A polymorphs and processes to prepare them.

### CROSS REFERENCE TO RELATED APPLICATIONS

This application is a Utility patent application which claims priority from Provisional Application Ser. No. 61/258,799, entitled “REBAUDIOSIDE A POLYMORPHS AND METHODS TO PREPARE THEM,” filed Nov. 6, 2009, and which is incorporated herein by reference in its entirety.

### FIELD OF THE INVENTION

This invention relates generally to polymorphic and/or amorphous forms of rebaudioside A and methods for preparing polymorphic and/or amorphous forms of rebaudioside A. More particularly, this invention relates to polymorphic and amorphous forms of rebaudioside A having consistent physical properties that are not affected by atmospheric moisture.

### BACKGROUND OF THE INVENTION

Stevia is a genus of about 240 species of herbs and shrubs in the sunflower family (Asteraceae), native to subtropical and tropical South America and Central America.

The species Stevia rebaudiana Bertoni, commonly known as sweet leaf, sugarleaf, or simply stevia, is widely grown for its sweet leaves. The leaves have traditionally been used as a sweetener. Steviosides and rebaudiosides are the major constituents of glycosides found in the leaves of the stevia plant.

Stevia extracts generally contain a high percentage of the glycosides of the diterpene steviol. The leaves of stevia rebaudiana contain 10 different steviol glycosides. Steviol glycosides are considered high intensity sweeteners (about 250-300 times that of sucrose) and have been used for several years in a number of countries as a sweetener for a range of food products. Stevioside and rebaudioside A are the principal sweetening compounds and generally accompanied by smaller amounts of other steviol glycosides. The taste quality of rebaudioside A is better than stevioside, because of increased sweetness and decreased bitterness (Phytochemistry 68, 2007, 1855-1863).

The structures and chemical abstract service registry numbers for steviol and its glycosides that are the main sweetening agents of the additive steviol glycosides are shown below:

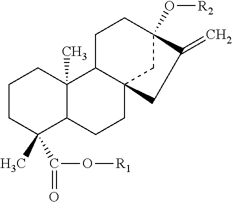

| | Compound | | | |
|---|---|---|---|---|
| | name | C. A. S. No. | R₁ | R₂ |
| 1 | Steviol | 471-80-7 | H | H |
| 2 | Steviolbioside | 41093-60-1 | H | β-Glc-β-Glc(2→1) |
| 3 | Stevioside | 57817-89-7 | β-Glc | β-Glc-β-Glc(2→1) |
| 4 | Rebaudioside | 58543-16-1 | β-Glc | β-Glc-β-Glc(2→1) |
| | A | | | \| |
| | | | | β-Glc(3→1) |
| 5 | Rebaudioside | 58543-17-2 | H | β-Glc-β-Glc(2→1) |
| | B | | | \| |
| | | | | β-Glc(3→1) |
| 6 | Rebaudioside | 63550-99-2 | β-Glc | β-Glc-β-Rha(2→1) |
| | C | | | \| |
| | | | | β-Glc(3→1) |
| 7 | Rebaudioside | 63279-13-0 | β-Glc-β-Glc(2→1) | β-Glc-β-Glc(2→1) |
| | D | | | \| |
| | | | | β-Glc(3→1) |
| 8 | Rebaudioside | 63279-14-1 | β-Glc-β-Glc(2→1) | β-Glc-β-Glc(2→1) |
| | E | | | |
| 9 | Rebaudioside | 438045-89-7 | β-Glc | β-Glc-β-Xyl(2→1) |
| | F | | | \| |
| | | | | β-Glc(3→1) |
| 10 | Rubusoside | 63849-39-4 | β-Glc | β-Glc |
| 11 | Dulcoside | 64432-06-0 | β-Glc | β-Glc-α-Rha(2→1) |
| | A | | | |

Steviol glycoside preparations are generally white to light yellow powders that are freely soluble in water and ethanol. The powders can be odorless or have a slight characteristic odor. Aqueous solutions are 200 to 300 times sweeter than sucrose under identical conditions. With its extracts having up to 300 times the sweetness of sugar, stevia has garnered attention with the rise in demand for low-carbohydrate, low-sugar food alternatives.

Medical research has also shown possible benefits of stevia in treating obesity and high blood pressure. Because stevia has a negligible effect on blood glucose, it is attractive as a natural sweetener to people on carbohydrate-controlled diets.

Therefore, a need exists for a sweetener that overcomes one or more of the current disadvantages noted above.

### BRIEF SUMMARY OF THE INVENTION

The present invention provides processes to prepare polymorphs or amorphous compositions of rebaudioside A where the degree of solvation or hydration is predictable and consistent.

The present invention provides a substantially pure rebaudioside A, polymorphic and amorphous forms of rebaudioside A, methods for purifying rebaudioside A, and methods for making polymorphic and amorphous forms of rebaudioside A.

In a particular embodiment, the method for purifying rebaudioside A comprises a simple crystallization. In one embodiment, a method for purifying rebaudioside A comprises the steps of combining crude rebaudioside A and an organic solvent or an aqueous organic solvent or water to form a glycosidic solution, the aqueous/organic solution comprising a solvent in an amount from about 0% to about 20% by weight, and crystallizing from the crude glycosidic solution, in a single step, a substantially pure rebaudioside A in a purity greater than about 95% by weight on a dry basis, wherein the polymorphic or amorphorous form is predictable and consistent.

For example, a rebaudioside A polymorph with between about 0.2 to about 0.5 moles of water to 1 mole of rebaudioside A has been prepared The isolated materials are free flowing. The isolated rebaudioside A polymorph has low solubility in water, being about 1.5 milligrams per milliliter (mg/ml) at 25° C.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1 depicts a DSC-TGA graph of polymorphic rebaudioside A from Example 1.

FIG. 2 depicts a DSC-TGA graph of polymorphic rebaudioside A from Example 2.

FIG. 3 depicts a DSC-TGA graph of polymorphic rebaudioside A from Example 3.

FIG. 4 depicts a DSC-TGA graph of polymorphic rebaudioside A from Example 4.

### DETAILED DESCRIPTION

In the specification and in the claims, the terms “including” and “comprising” are open-ended terms and should be interpreted to mean “including, but not limited to . . . . ” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The phrase “steviol glycosides” is recognized in the art and is intended to include the major and minor constituents of stevia. These include, but are not limited to components of stevia such as Steviol, Steviolbioside, Stevioside, Rebaudioside A, Rebaudioside B, Rebaudioside C, Rebaudioside D, Rebaudioside E, Rebaudioside F, Rubusoside and Dulcoside A. Typically, stevia contains less than a trace amount of rebaudioside D. Typical stevia extracts also contain less than about 0.1% by weight of rebaudioside D.

As used herein, the term “substantially pure steviol glycoside” refers to a steviol glycoside composition that includes at least about 80% by weight of steviol glycoside on a dry basis. In another aspect, the substantially or substantially pure steviol glycoside compositions include at least about 85% by weight, at least about 90% by weight, at least about 95% by weight, or at least about 98% by weight of steviol glycoside on a dry basis.

It is important to note that the polymorphs and/or amorphorous forms of the rebaudioside A described herein are unique in that the present polymorphs and/or amorphorous forms of rebaudioside A are stable under ambient conditions and remain free flowing. The polymorphic form of rebaudioside A of the present invention has a solubility in water of about 1.5 mg/ml at 25° C.

Up until this discovery, it has not been possible to accurately determine the percentage water present in the stevioside material. This is especially true with steviol materials that were “dried” (under vacuum and/or elevated temperatures) as once exposed to the environment, the material would immediately absorb moisture, making it difficult to know the amount of stevioside present and whether the material was crystalline, amorphorous and/or one or more polymorphs.

As used herein, the term “substantially pure form” refers to a steviol glycoside composition that includes at least about 80% by weight of a particular polymorphic or amorphous form of steviol glycoside. In another aspect, the substantially pure form of a steviol glycoside composition includes at least about 85% by weight, at least about 90% by weight, at least about 95% by weight, or at least about 98% by weight of a particular steviol glycoside polymorphic or amorphous form.

It has been discovered that a polymorphic forms of rebaudioside A results from using the purification method described herein below, including: Form 1: a rebaudioside A hydrate. Those of ordinary skill in the art will appreciate that the aqueous solution and the temperatures of the purification processes described herein influence the resulting polymorphs in a substantially pure rebaudioside A composition.

Polymorphism is defined as the ability of a substance to exist as two or more crystalline states that have different arrangements and/or conformations of the molecules in the crystal lattice. Polymorphism may cause physical properties such as density, melting point, and rate of dissolution to change.

The rebaudioside A of the invention can be used in beverages, broths, and beverage preparations selected from the group comprising carbonated, non-carbonated, frozen, semi-frozen (“slush”), non-frozen, ready-to-drink, concentrated (powdered, frozen, or syrup), dairy, non-dairy, herbal, non-herbal, caffeinated, non-caffeinated, alcoholic, non-alcoholic, flavored, non-flavored, vegetable-based, fruit-based, root/tuber/corn-based, nut-based, other plant-based, cola-based, chocolate-based, meat-based, seafood-based, other animal-based, algae-based, calorie enhanced, calorie-reduced, and calorie-free products, optionally dispensed in open containers, cans, bottles or other packaging. Such beverages and beverage preparations can be in ready-to-drink, ready-to-cook, ready-to-mix, raw, or ingredient form and can use the rebaudioside A (polymorphs/amorphous forms) of the invention as a sole sweetener or as a co-sweetener.

The rebaudioside A of the invention can be used in foods and food preparations (e.g. sweeteners, soups, sauces, flavorings, spices, oils, fats, and condiments) from dairy-based, cereal-based, baked, vegetable-based, fruit-based, root/tuber/corm-based, nut-based, other plant-based, egg-based, meat-based, seafood-based, other animal-based, algae-based, processed (e.g. spreads), preserved (e.g. meals-ready-to-eat rations), and synthesized (e.g. gels) products. Such foods and food preparations can be in ready-to-eat, ready-to-cook, ready-to-mix, raw, or ingredient form and can use the steviol glycoside polymorphs and/or amorphous forms as a sole sweetener or as a co-sweetener.

The rebaudioside A of the invention can be used in candies, confections, desserts, and snacks selected from the group comprising dairy-based, cereal-based, baked, vegetable-based, fruit-based, root/tuber/corm-based, nut-based, gum-based, other plant-based, egg-based, meat-based, seafood-based, other animal-based, algae-based, processed (e.g. spreads), preserved (e.g. meals-ready-to-eat rations), and synthesized (e.g. gels) products. Such candies, confections, desserts, and snacks can be in ready-to-eat, ready-to-cook, ready-to-mix, raw, or ingredient form, and can use the steviol glycoside polymorphs and/or amorphous forms as a sole sweetener or as a co-sweetener.

The rebaudioside A of the invention can be used in prescription and over-the-counter pharmaceuticals, assays, diagnostic kits, and therapies selected from the group comprising weight control, nutritional supplement, vitamins, infant diet, diabetic diet, athlete diet, geriatric diet, low carbohydrate diet, low fat diet, low protein diet, high carbohydrate diet, high fat diet, high protein diet, low calorie diet, non-caloric diet, oral hygiene products (e.g. toothpaste, mouthwash, rinses, floss, toothbrushes, other implements), personal care products (e.g. soaps, shampoos, rinses, lotions, balms, salves, ointments, paper goods, perfumes, lipstick, other cosmetics), professional dentistry products in which taste or smell is a factor (e.g. liquids, chewables, inhalables, injectables, salves, resins, rinses, pads, floss, implements), medical, veterinarian, and surgical products in which taste or smell is a factor (e.g. liquids, chewables, inhalables, injectables, salves, resins, rinses, pads, floss, implements), and pharmaceutical compounding fillers, syrups, capsules, gels, and coating products.

The rebaudioside A of the invention can be used in consumer goods packaging materials and containers selected from the group comprising plastic film, thermoset and thermoplastic resin, gum, foil, paper, bottle, box, ink, paint, adhesive, and packaging coating products.

The rebaudioside A of the invention can be used in goods including sweeteners, co-sweeteners, coated sweetener sticks, frozen confection sticks, medicine spoons (human and veterinary uses), dental instruments, pre-sweetened disposable tableware and utensils, sachets, edible sachets, potpourris, edible potpourris, artificial flowers, edible artificial flowers, clothing, edible clothing, massage oils, and edible massage oils.

The rebaudioside A of the invention can also be used with “artificial sweeteners”. Artificial sweeteners are those, other than sucrose, such as cyclamates and salts thereof, sucralose, aspartame, saccharin and salts thereof, stevia (Truvia™), rebaudioside A, xylitol, acesulfame-K and the like.

The following paragraphs enumerated consecutively from 1 through 23 provide for various aspects of the present invention. In one embodiment, in a first paragraph (1), the present invention provides a substantially pure rebaudioside A composition comprising a hydrate of rebaudioside A.

2. A substantially pure rebaudioside A composition according to paragraph 1, further comprising a molar percentage of absorption moisture.

3. A substantially pure rebaudioside A composition according to paragraph 1 or 2, wherein the molar ratio of bound moisture to rebaudioside A is between above zero and about 1.

4. A substantially pure rebaudioside A composition comprising rebaudioside A and a mole percentage of water.

5. The substantially pure rebaudioside A according to paragraph 4, wherein the mole ratio of crystalline water to rebaudioside A is between above zero to about 50.

6. The substantially pure rebaudioside A according to paragraph 5, wherein the mole ratio of crystalline water to rebaudioside A is between above zero to about 10.

7. The substantially pure rebaudioside A according to paragraph 6, wherein the mole ratio of crystalline water to rebaudioside A is between above zero to about 5.

8. The substantially pure rebaudioside A according to paragraph 7, wherein the mole ratio of crystalline water to rebaudioside A is between above zero to about 1.

9. The substantially pure rebaudioside A according to paragraph 8, wherein the mole ratio of crystalline water to rebaudioside A is from about 0.01 to about 0.5.

10. The substantially pure rebaudioside A according to paragraph 9, wherein the mole ratio of crystalline water to rebaudioside A is from about 0.1 to about 0.5.

11. The substantially pure rebaudioside A according to paragraph 10, wherein the mole ratio of crystalline water to rebaudioside A is from about 0.2 to about 0.5.

12. A composition comprising the substantially pure rebaudioside A according to paragraph 4 and further comprising rebaudioside D.

13. The substantially pure rebaudioside A according to paragraph 12, wherein the mole ratio of water to rebaudioside A is from about 0.01 and about 3.

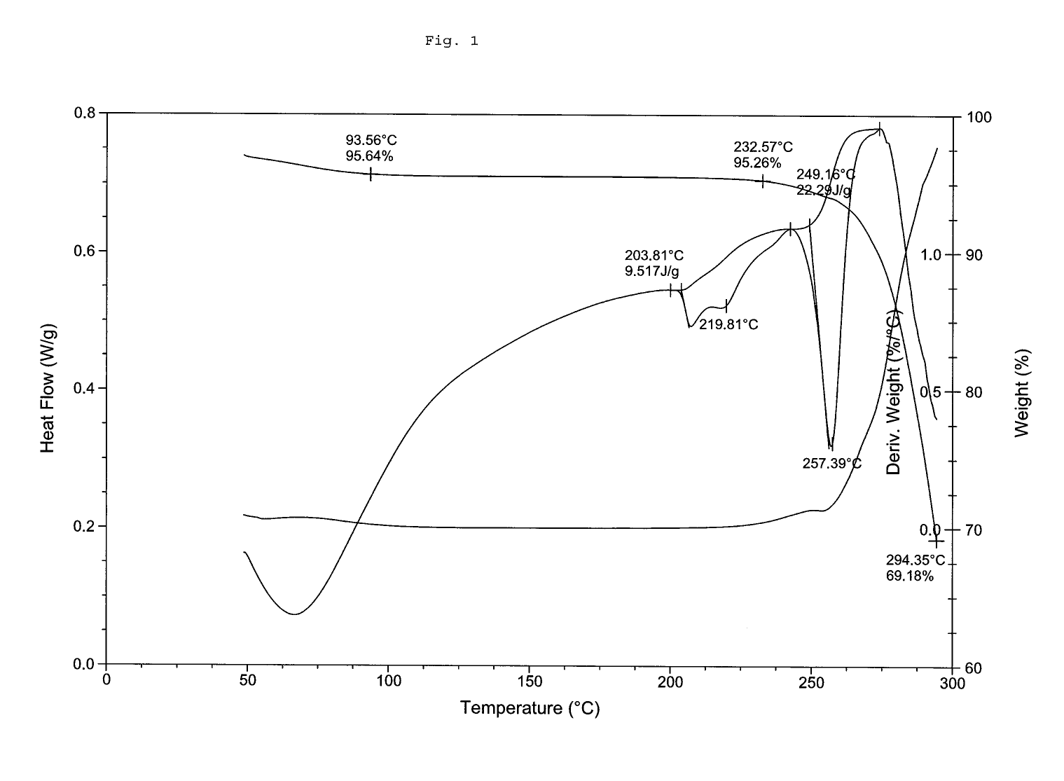

14. The substantially pure rebaudioside A according to paragraph 4, wherein the DSC-TGA profile of the polymorphic form of the rebaudioside A is depicted by FIG. 1, 2, 3 or 4.

15. The substantially pure rebaudioside A according to paragraph 4, wherein the solubility of the rebaudioside A in water at 25° C. is about 1.5 mg/ml.

16. The substantially pure rebaudioside A of paragraph 4, wherein the equilibrium solubility is from about 0.1% to about 0.8% by weight in water at ambient temperature.

17. The substantially pure rebaudioside A of paragraph 13, wherein the equilibrium solubility is from about 0.2% to about 0.8%, more particularly from about 0.2% to about 0.7%, more particularly from about 0.2% to about 0.6% and most particularly from about 0.15% to about 0.2% in water by weight percent at ambient temperature.

18. A method to prepare a rebaudioside A polymorph comprising the steps:

adding an amount of substantially pure form of rebaudioside A to 100 g of water until dissolution, in particular complete dissolution, occurs to form a solution; optionally the solution can be heated up to about 70° C. to effect dissolution, in particular from about 1 g to about 40 g of RA, in particular from about 2 g to about 30 g, and more particularly from about 5 g to about 20 g and most particularly about 20 g per 100 g of water,

allowing the solution to remain at room temperature until a precipitate is formed; and

collecting the precipitate to provide a polymorph of rebaudioside A.

19. The paragraph of claim 18, wherein the polymorph of rebuadioside A is dried.

20. The method of paragraph 19, wherein the polymorph of rebaudioside A is dried at an elevated temperature below the melting point of the polymorph, for example, up to about 250 to about 255° C.

21. The method of paragraph 20, wherein the elevated temperature is from about 30° C. to about 150° C.

22. The method of any of paragraphs 19 through 21, wherein the polymorph is dried under reduced pressure.

23. The product of any of paragraphs 18 through 22.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

### EXAMPLES

### Example 1

2 g of rebaudioside A (commercial RA, 97% measured by HPLC) was mixed with 10 mL of water, and the mixture was stirred at room temperature until the solid was totally dissolved. The solution was then allowed to remain at room temperature, without stirring for about 5 hours. After about 5 hours, a precipitate was observed. A particulate/crystalline material precipitate was collected and dried at 100° C. for 2 hours at atmospheric pressure to yield a hydrate of RA. The hydrate contained 4.94% of water by weight as determined by DSC-TGA. FIG. 1 is a DSC-TGA graphical representation of the crystalline form of rebaudioside A of Example 1.

Referring to FIG. 1, under 93.56° C., the weight ratio of residual hydrate is 95.64%, which demonstrates the loss of 4.56% as free water. Under 232.57° C., the residue is 95.26%, with water also lost under this temperature. Therefore, the percentage of crystalline water is 95.64-95.26=0.38% (0.214 mole water/1 mole RA). The different thermal potential energies indicate two different forms of water. This is an evidence of water crystallized within the rebaudioside A polymorphic structure.

Parameters for the DSC-TGA determination were as follows: Temperature gradient: 10 degree/min.; Temperature range: 0-300° C.; Instrument type: SDT Q600 V20.5 Build 15.

The material was a free flowing particulate.

### Example 2

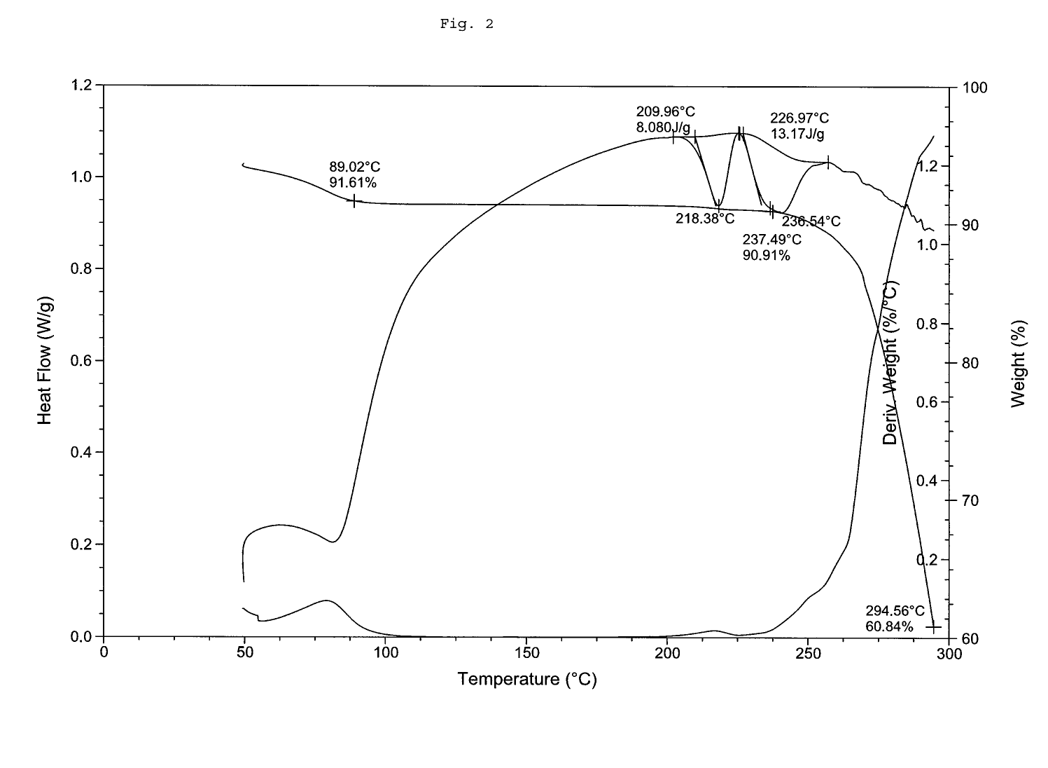

The procedure according to Example 1 was followed, except dissolution was performed at 65° C. The remaining other steps were the same as Example 1. A paste was formed, filtered and the resulting particles were dried at 100° C. for 2 hours at atmospheric pressure to yield a hydrate of RA. The hydrate contained 9.09% of water by weight as determined by DSC-TGA. FIG. 2 is a DSC-TGA graphical representation of the crystalline form of rebaudioside A of Example 2.

Referring to FIG. 2, under 89.02° C., the weight ratio of residual hydrate is 91.61%, which demonstrates the loss of 8.39% as free water. Under 237.49° C., the residue is 90.91%, with water also lost under this temperature. Therefore, the percentage of crystalline water is 91.61-90.91=0.70% (0.414 mole water/1 mole RA). The different thermal potential energies indicate two different forms of water. This is evidence of water crystallized within the rebaudioside A polymorphic structure.

Parameters for the DSC-TGA determination were as follows: Temperature gradient: 10 degree/min.; Temperature range: 0-300° C.; Instrument type: SDT Q600 V20.5 Build 15.

The material was a free flowing particulate.

### Example 3

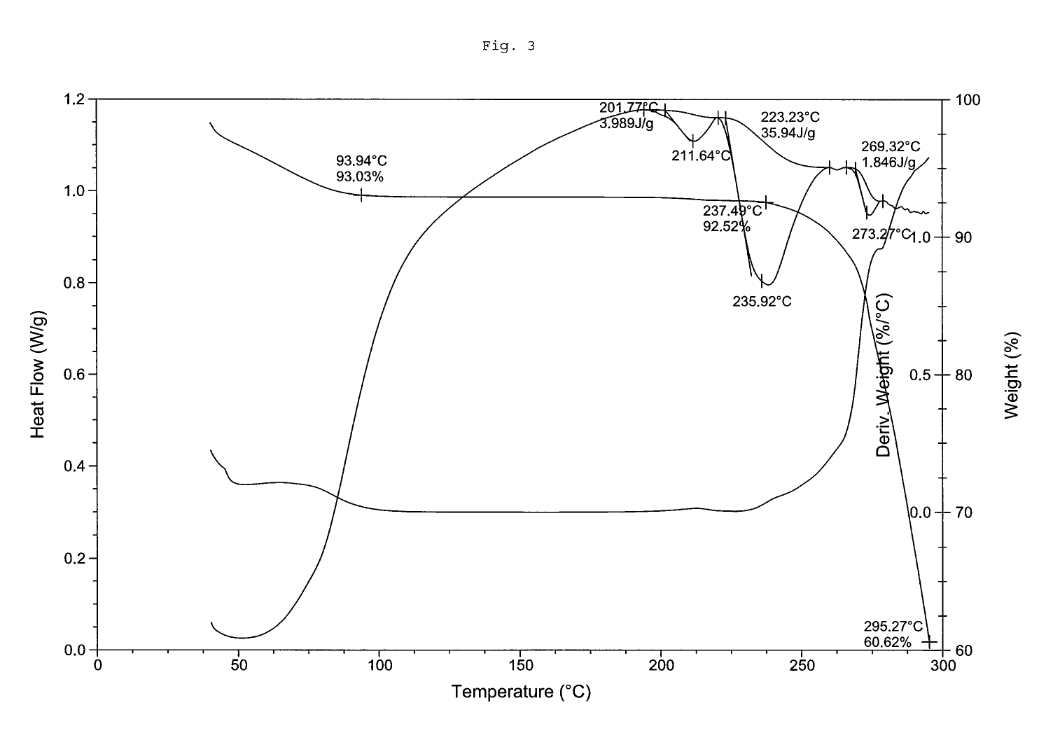

The procedure according to Example 1 was followed, except 3 g of rebaudioside A was used. The remaining other steps were the same as Example 1. A paste was formed, filtered and the resulting particles were dried at 100° C. for 2 hours at atmospheric pressure to yield a hydrate of RA. The hydrate contained 7.48% of water by weight as determined by DSC-TGA. FIG. 3 is a DSC-TGA graphical representation of the crystalline form of rebaudioside A of Example 3.

Referring to FIG. 3, under 93.94° C., the weight ratio of residual hydrate is 93.03%, which demonstrates the loss of 6.97% as free water. Under 237.49° C., the residue is 92.52%, with water also lost under this temperature. Therefore, the percentage of crystalline water is 93.03-92.52=0.51% (0.296 mole water/1 mole RA). The different thermal potential energies indicate two different forms of water. This is evidence of water crystallized within the rebaudioside A polymorphic structure.

Parameters for the DSC-TGA determination were as follows: Temperature gradient: 10 degree/min.; Temperature range: 0-300° C.; Instrument type: SDT Q600 V20.5 Build 15.

The material was a free flowing particulate.

### Example 4

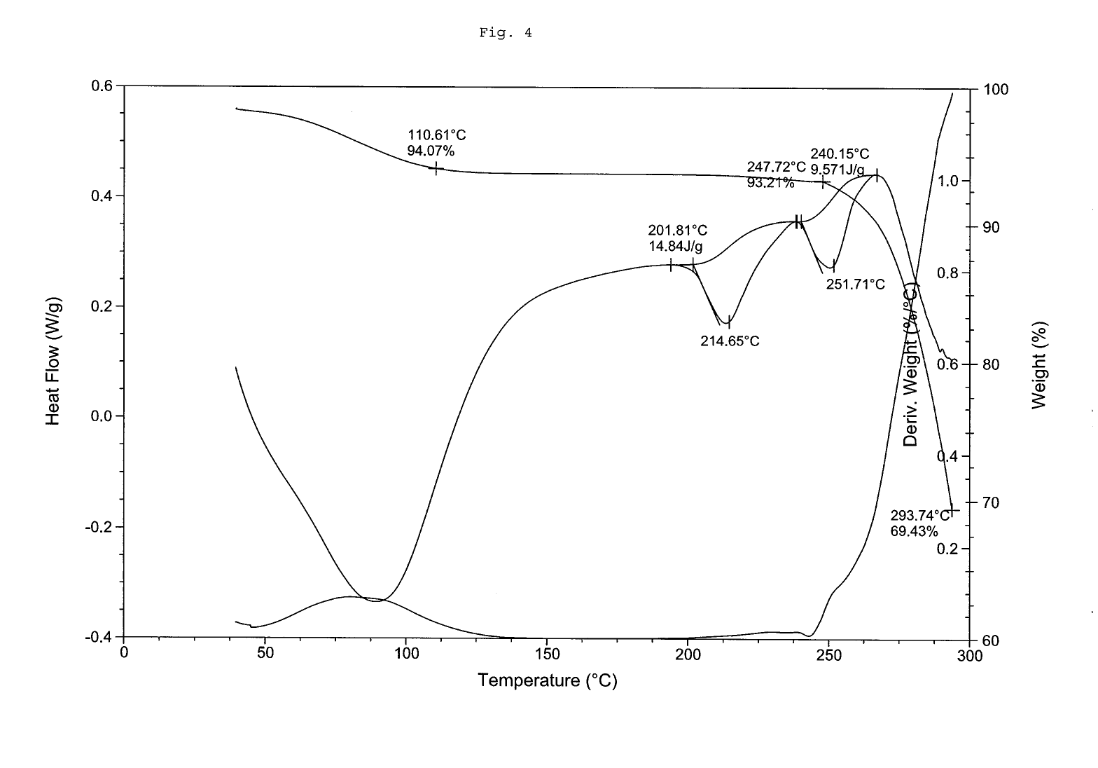

The procedure according to Example 1 was followed, except 4 g of rebaudioside A was used. The remaining other steps were the same as Example 1. A paste was formed, filtered and the resulting particles were dried at 100° C. for 2 hours at atmospheric pressure to yield a hydrate of RA. The hydrate contained 6.79% of water by weight as determined by DSC-TGA. FIG. 4 is a DSC-TGA graphical representation of the crystalline form of rebaudioside A of Example 4.

Referring to FIG. 4, under 110.61° C., the weight ratio of residual hydrate is 94.07%, which demonstrates the loss of 5.93% as free water. Under 247.72° C., the residue is 93.21%, with water also lost under this temperature. Therefore, the percentage of crystalline water is 94.07−93.21=0.86% (0.496 mole water/1 mole RA). The different thermal potential energies indicate two different forms of water. This is evidence of water crystallized within the rebaudioside A polymorphic structure.

Parameters for the DSC-TGA determination were as follows: Temperature gradient: 10 degree/min.; Temperature range: 0-300° C.; Instrument type: SDT Q600 V20.5 Build 15.

The material was a free flowing particulate.

General Procedure for the Preparation of the RA Polymorphs:

Dissolve a quantity of substantially pure RA in water with or without heating. In one embodiment the RA is completely dissolved in water. Suitable ranges of RA are from about 1 g to about 40 g of RA/100 ml water, in particular from about 2 g to about 30 g, and more particularly from about 5 g to about 20 g and most particularly about 20 g per 100 g of water.

Optionally, the solution can be heated from about 20° C. to about 70° C. to help effect dissolution of the solid RA.

After the solid material is dissolved, the solution is permitted to cool, if necessary, and remain at room temperature (approximately 25° C.) until a precipitate is formed. The precipitate is then collected and dried below the melting point of RA. Generally, the material is heated below from about 250 to about 255° C., more particularly at about 150° C. and most particularly at about 100° C. until a constant weight is achieved. Drying can be conducted under reduced pressure to help speed the drying process.

Method to Calculate Molar Ratio Percentages:

Based on example 1, the following can be determined:

free water: 4.56% by weight

crystalline water: 0.38% by weight

Total RA: 95.26% by weight

molecular weight of RA: 967.01

molecular weight of water: 18

Therefore, the molar ratio of crystalline water to RA=(0.38/18)/(95.26/967.01)=0.214

In similar fashion, the molar ratio of free water to RA=(4.56/18)/(95.26/967.01)=2.572.

Equilibrium Studies

An equilibrium solubility refers to the long term solubility of amorphous rebaudioside A. Greater than 40% by weight of amorphous RA can be dissolved in water at room temperature (based on 100 g of water). However, when the solution is left for a period of approximately 12 hours, the majority of the rebaudioside A has precipitated out of solution. By progressively lowering the concentration of rebaudioside A initially dissolved in water, an equilibrium solubility is the concentration at which no RA precipitates from solution.

The initial solubility of amorphous RA in water is above 40%. 2.9783 g RA was dissolved in 6.5 ml of water, and the solubility was measured to be 45.8%. Meanwhile, 0.0646 g RA hydrate from Example 1 was dissolved in 32 ml water and the equilibrium solubility was measured to be 0.2%. The material remained dissolved in the water at ambient temperature for at least 7 days.

Various Moisture Molar Ratios in Hydrates

| Example | Bound moisture* | Absorption moisture** | All moisture*** |
|---|---|---|---|
| 1 | 0.214 | 2.573 | 2.787 |
| 2 | 0.414 | 4.958 | 5.372 |
| 3 | 0.296 | 4.047 | 4.343 |
| 4 | 0.496 | 3.418 | 3.914 |

*represents the amount of crystalline water in hydrate.
**represents the amount of amorphous water in composition in the hydrate.
***Represents the amount of all water including crystalline and amorphous water in the hydrate.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A substantially pure rebaudioside A composition comprising a hydrate of rebaudioside A.

2. A substantially pure rebaudioside A composition according to claim 1, further comprising a molar percentage of absorption moisture.

3. A substantially pure rebaudioside A composition according to claim 1, wherein the molar ratio of bound moisture to rebaudioside A is between above zero and about 1.

4. A substantially pure rebaudioside A composition comprising rebaudioside A and a mole percentage of water.

5. The substantially pure rebaudioside A according to claim 4, wherein the mole ratio of crystalline water to rebaudioside A is between above zero to about 50.

6. The substantially pure rebaudioside A according to claim 5, wherein the mole ratio of crystalline water to rebaudioside A is between above zero to about 10.

7. The substantially pure rebaudioside A according to claim 6, wherein the mole ratio of crystalline water to rebaudioside A is between above zero to about 5.

8. The substantially pure rebaudioside A according to claim 7, wherein the mole ratio of crystalline water to rebaudioside A is between above zero to about 1.

9. The substantially pure rebaudioside A according to claim 8, wherein the mole ratio of crystalline water to rebaudioside A is from about 0.01 to about 0.5.

10. The substantially pure rebaudioside A according to claim 9, wherein the mole ratio of crystalline water to rebaudioside A is from about 0.1 to about 0.5.

11. The substantially pure rebaudioside A according to claim 10, wherein the mole ratio of crystalline water to rebaudioside A is from about 0.2 to about 0.5.

12. A composition comprising the substantially pure rebaudioside A according to claim 4 and further comprising rebaudioside D.

13. The substantially pure rebaudioside A according to claim 12, wherein the mole ratio of water to rebaudioside A is from about 0.01 and about 3.

14. The substantially pure rebaudioside A according to claim 4, wherein the DSC-TGA profile of the polymorphic form of the rebaudioside A is depicted by FIG. 1, 2, 3 or 4.

15. The substantially pure rebaudioside A according to claim 4, wherein the solubility of the rebaudioside A in water at 25° C. is about 1.5 mg/ml.

16. The substantially pure rebaudioside A of claim 4, wherein the equilibrium solubility is from about 0.2% to about 0.8% by weight in water at ambient temperature.

17. The substantially pure rebaudioside A of claim 13, wherein the equilibrium solubility is from about 0.2% to about 0.5% in water by weight at ambient temperature.

18. A method to prepare a rebaudioside A polymorph comprising the steps:
dissolving an amount of substantially pure form of rebaudioside A in 100 g of water until complete dissolution occurs to form a solution;
allowing the solution to remain at room temperature until a precipitate is formed; and
collecting the precipitate to provide a polymorph of rebaudioside A.

19. The method of claim 18, wherein the polymorph of rebuadioside A is dried.

20. The method of claim 19, wherein the polymorph of rebaudioside A is dried at an elevated temperature below the melting point of the polymorph.

21. The method of claim 20, wherein the elevated temperature is about 100° C.

22. The method of claim 21, wherein the polymorph is dried under reduced pressure.

23. The product of claim 18.

24. The product of claim 19.

25. The product of claim 20.
